(19) United States
(12) Patent Application Publication
(10) Pub. No.: US 2021/0402183 A1
(43) Pub. Date: Dec. 30, 2021

(54) Electrostimulation Device

(71) Applicant: Nippon Telegraph and Telephone Corporation, Tokyo (JP)

(72) Inventor: Toshiki Wada, Tokyo (JP)

(21) Appl. No.: 17/291,011

(22) PCT Filed: Nov. 27, 2019
(86) PCT No.: PCT/JP2019/046372

§ 371 Date: May 4, 2021

(30) Foreign Application Priority Data
Dec. 11, 2018 (JP) 2018-231358

(51) Int. Cl. A61N 1/36; G08B 6/00

(52) U.S. Cl. CPC A61N 1/36034; G08B 6/00

(57) ABSTRACT

An output selection unit is provided between one or more output units and a plurality of electrodes. The output selection unit is configured to freely switch a combination of the electrodes constituting an electrode pair connected to the output unit. A control unit sends a selection signal to the output selection unit to indicate the combination of the electrodes constituting the electrode pair connected to the output unit.

### CROSS-REFERENCE TO RELATED APPLICATIONS

This application is a national phase entry of PCT Application No. PCT/JP2019/046372, filed on Nov. 27, 2019, which claims priority to Japanese Application No. 2018-231358, filed on Dec. 11, 2018, which applications are hereby incorporated herein by reference.

### TECHNICAL FIELD

The present invention relates to an electrical stimulation apparatus, and particularly to an electrical stimulation apparatus that stimulates a human body by passing an electrical current between electrodes.

### BACKGROUND

A low-frequency therapy apparatus, an electrical muscle stimulation apparatus, and the like have been used as electrical stimulation apparatuses that stimulate a human body by passing an electrical current between electrodes.

FIG. 5 illustrates a main part of a low-frequency therapy apparatus described in Patent Literature 1 as an example of a conventional electrical stimulation apparatus. This low-frequency therapy apparatus includes a pair of pads 300 (300A, 300B) affixed to a treatment site. The pad 300A on a high potential side (positive side) is provided with electrodes a1, a2, and a3, and the pad 300B on a low potential side (negative side) is provided with electrodes b1, b2, and b3.

In this low-frequency therapy apparatus, the electrodes a1, a2, and a3 provided on the pad 300A are selectively set at a high potential, and the electrodes b1, b2, and b3 provided on the pad 300B are selectively set at a low potential. As a result, it is possible to apply a massaging stimulus, a rubbing stimulus, and the like by changing the position or area of the energization area.

For example, as illustrated in FIG. 6A, when the electrode a1 is set at a high potential and the electrode b1 is set at a low potential, a current flows from the electrode a1 to the electrode b1. From this state, as illustrated in FIG. 6B, when the electrode b2, in place of the electrode b1, is set at a low potential, a current flows from the electrode a1 to the electrode b2. This changes the position of the energization area (effective electrode arrangement).

For example, as illustrated in FIG. 7A, when the electrodes a1 and a2 are set at a high potential and the electrodes b1 and b2 are set at a low potential, currents flow from the electrodes a1 and a2 to the electrodes b1 and b2. From this state, as illustrated in FIG. 7B, when the electrode a3 is set at a high potential and the electrode b3 is set at a low potential, a current also flows from the electrode a3 to the electrode b3. This changes the area of the energization area (effective electrode area).

### CITATION LIST

### Patent Literature

Patent Literature 1: JP 2008-125694 A

### Non Patent Literature

Non Patent Literature 1: Kajimoto et al. “Forehead Electrotactile Display”, collected papers of the 11th conference of the Virtual Reality Society, Japan, pp. 1-4, 2006.

### SUMMARY

### Technical Problem

However, according to the technology disclosed in Patent Literature 1, the direction of each current flow can be changed to only a direction from one of the electrodes a1, a2, and a3 to one of the electrodes b1, b2, and b3. For example, as illustrated in FIG. 8, a current flow from the electrode a1 to the electrode b1 cannot be changed to a current flow from the electrode b1 to the electrode a1. In addition, for example, as illustrated in FIG. 9, a current flow from the electrode b1 to the electrode b3 cannot be added, in addition to a current flow from the electrode a2 to the electrode b2.

In this manner, according to the technology disclosed in Patent Literature 1, it is impossible to produce the current flow from the electrode b1 to the electrode a1 and the current flow from the electrode b1 to the electrode b3 to diversify a stimulus to a human body. This means that, even when the technology disclosed in Patent Literature 1 is utilized in the field of tactile presentation via electrical stimulation as disclosed in Non Patent Literature 1, tactile presentation cannot be diversified.

Embodiments of the present invention are devised to solve such a problem, and an object of embodiments of the present invention is to provide an electrical stimulation apparatus capable of diversifying tactile presentation in the field of tactile presentation via electrical stimulation.

### Means for Solving the Problem

To attain the above-mentioned object, embodiments of the present invention include a plurality of electrodes (1), one or more output units (2), each of the one or more output units (2) including a first output end (P1) and a second output end (P2), the first and second output ends being different in potential, and an output selection unit (3) configured to freely switch a combination of the electrodes constituting an electrode pair connected between the first output end and the second output end of the output unit.

According to embodiments of the present invention, the combination of the electrodes constituting the electrode pair connected between the first output end and the second output end of the output unit (hereinafter referred to simply as the electrode pair connected to the output unit) can be freely switched. As a result, the combination of the electrodes constituting the electrode pair connected to the output unit can be dynamically switched to freely change the number of electrodes connected to the output unit (effective electrode area) and the positions of the electrodes connected to the output unit (effective electrode arrangement).

As a configuration example of embodiments of the present invention, the control unit may indicate, to the output selection unit, the combination of the electrodes constituting the electrode pair connected to the output unit, thereby changing the number of electrodes connected to the output unit and the positions of the electrodes connected to the output unit.

Further, as a configuration example of embodiments of the present invention, the control unit may include a function unit that inspects a contact state between each of the plurality of electrodes and a place where the electrode is disposed, and excludes an electrode determined to be in a poor contact state from the combination of the electrodes indicated to the output selection unit.

Note that, in the above description, by way of example, components in the drawings, which correspond to the components of embodiments of the invention, are indicated by reference numerals in parentheses.

### Effects of Embodiments of the Invention

As described above, according to embodiments of the present invention, because the combination of the electrodes constituting the electrode pair connected to the output unit can be freely switched, the number of electrodes connected to the output unit (effective electrode area) and the positions of the electrodes connected to the output unit (effective electrode arrangement) can be freely changed to diversify tactile presentation in the field of tactile presentation via electrical stimulation.

### BRIEF DESCRIPTION OF THE DRAWINGS

FIG. 1 is a diagram illustrating a main part of an electrical stimulation apparatus according to an embodiment of the present invention.

FIG. 2 is a diagram illustrating a specific example 1 of an operation of an output selection unit according to a selection signal from a control unit in the electrical stimulation apparatus.

FIG. 3 is a diagram illustrating a specific example 2 of an operation of the output selection unit according to a selection signal from the control unit in the electrical stimulation apparatus.

FIG. 4 is a diagram illustrating an example in which the control unit is provided with a contact state inspection unit.

FIG. 5 is a diagram illustrating a main part of a low-frequency therapy apparatus described in Patent Literature 1.

FIG. 6A is a diagram illustrating an example in which the position of an energization area is changed in the low-frequency therapy apparatus.

FIG. 6B is a diagram illustrating another example in which the position of an energization area is changed in the low-frequency therapy apparatus.

FIG. 7A is a diagram illustrating an example in which the area of the energization area is changed in the low-frequency therapy apparatus.

FIG. 7B is a diagram illustrating an example in which the area of the energization area is changed in the low-frequency therapy apparatus.

FIG. 8 is a diagram illustrating an example in which a current flow between electrodes cannot be produced in the low-frequency therapy apparatus.

FIG. 9 is a diagram illustrating another example in which a current flow between electrodes cannot be produced in the low-frequency therapy apparatus.

### DETAILED DESCRIPTION OF ILLUSTRATIVE EMBODIMENTS

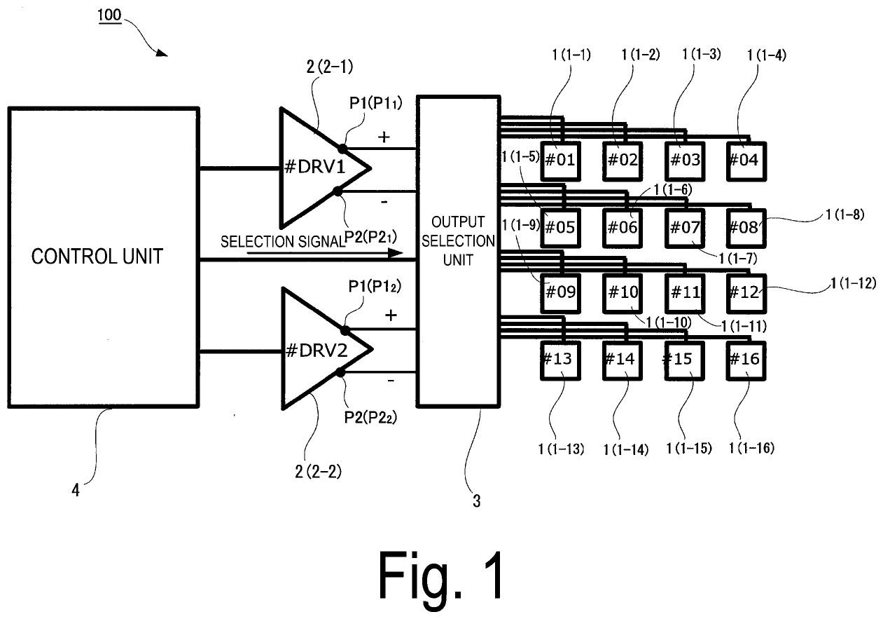

Hereinafter, an embodiment of the present invention will be described in detail. FIG. 1 is a diagram illustrating a main part of an electrical stimulation apparatus 100 according to the embodiment of the present invention.

The electrical stimulation apparatus 100 includes a plurality of electrodes 1 arranged in a matrix, two output units 2, an output selection unit 3 provided between the two output units 2 and the plurality of electrodes 1, and a control unit 4 that controls the entire apparatus.

The output units 2 each include an output end P1 on a high electric potential side (positive side) and an output end P2 on a low electric potential side (negative side), and operate under a command from the control unit 2. In the present embodiment, one of the two output units 2 is referred to as an output unit 2-1 (#DRV1), and the other is referred to as an output unit 2-2 (#DRV2). Hereinafter, the output end P1 is referred to as an output end on a high potential side and the output end P2 as an output end on a low potential side.

In the present embodiment, the plurality of electrodes 1 are 16 electrodes 1-1 to 1-16, and the electrodes 1-1 to 1-16 are independently arranged in four columns and four rows. The electrodes 1-1 to 1-16 are provided on a pad and attached to a user body. Hereinafter, numbers indicating the electrode 1-1 to 1-16 are denoted as #01 to #16.

In the present embodiment, the output selection unit 3 is configured to freely switch a combination of the electrodes 1 that constitute an electrode pair connected between the output end P1 (P1₁, P1₂) on the high potential side and the output end P2 (P2₁, P2₂) on the low potential side of the output unit 2 (2-1, 2-2). For example, the output selection unit 3 includes a lot of switching elements such as transistors, and can switch a connection mode of the switching elements to freely switch the combination of the electrodes 1 connected to the output end P1 (P1₁, P1₂) on the high potential side and the output end P2 (P2₁, P2₂) on the low potential side.

Further, in the present embodiment, the control unit 4 has a function of sending, to the output selection unit 3, a signal indicating the combination of the electrodes 1 connected to the output end P1 (P1₁, P1₂) on the high potential side and the output end P2 (P2₁, P2₂) on the low potential side of the output unit 2 (2-1, 2-2) (hereinafter, this signal is referred to as a selection signal).

In the present embodiment, the control unit 4 is enabled by hardware including a processor, a storage device, and so on, and a program that cooperates with the hardware to implement various functions. An operation of the output selection unit 3 according to the selection signal from the control unit 4 will be described below using specific examples. Note that in the case of presenting information such as characters on a display to the user, the control unit 4 outputs a signal indicating the information as the selection signal to the output selection unit 3.

### Specific Example 1

It is assumed that the control unit 4 sends, to the output selection unit 3, a selection signal indicating “#01, #02” as the combination of the electrodes 1 connected to the output end P1₁ on the high potential side of the output unit 2-1, and “#11, #12, #15, #16” as the combination of the electrodes 1 connected to the output end P2₁ on the low potential side of the output unit 2-1.

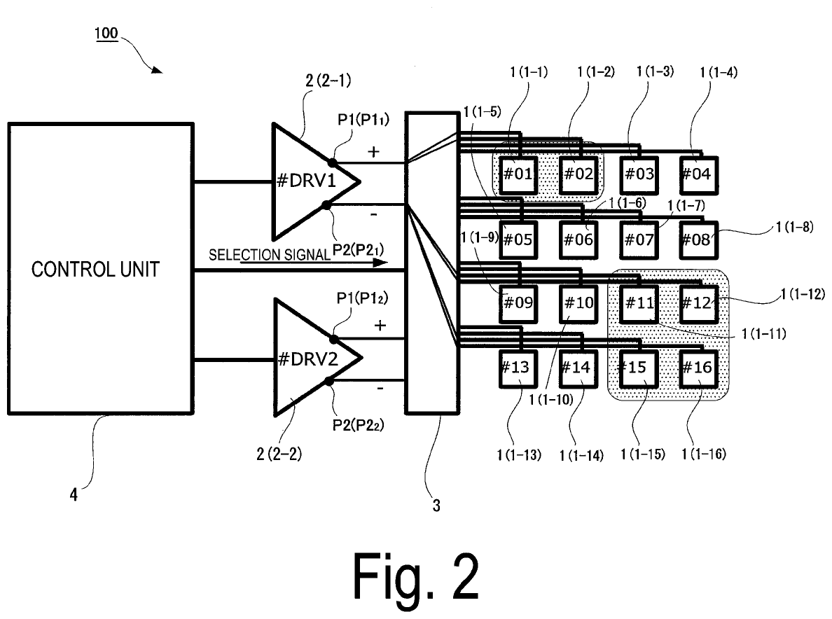

When the selection signal is sent, as illustrated in FIG. 2, the output selection unit 3 connects the electrodes 1-1 and 1-2 to the output end P1₁ on the high potential side of the output unit 2-1, and connects the electrodes 1-11, 1-12, 1-15, and 1-16 to the output end P2₁ on the low potential side of the output unit 2-1.

This causes the electrodes 1-1 and 1-2 to form an equipotential surface, such that the two electrodes 1-1 and 1-2 create one large positive electrode. In addition, the electrodes 1-11, 1-12, 1-15, and 1-16 form an equipotential surface, such that the four electrodes 1-11, 1-12, 1-15, and 1-16 create one large negative electrode.

That is, an electrode pair including the combination of the electrodes 1-1 and 1-2 as a positive electrode and the combination of the electrodes 1-11, 1-12, 1-15, and 1-16 as a negative electrode is created between the output end P1₁ on the high potential side and the output end P2₁ on the low potential side of the output unit 2-1, and a current flows to the electrode pair through the user body.

Note that the electrodes other than the selected electrodes are not connected to anywhere, and are in a floating state. Thus, these electrodes do not affect the output. The same applies to a specific example 2 below.

### Specific Example 2

Next, it is assumed that the control unit 4 sends, to the output selection unit 3, a selection signal indicating “#09” as the combination of the electrodes 1 connected to the output end P1₂ on the high potential side of the output unit 2-2, and “#14” as the combination of the electrodes 1 connected to the output end P2₂ on the low potential side of the output unit 2-2.

When the selection signal is sent, as illustrated in FIG. 3, the output selection unit 3 connects the electrode 1-9 to the output end P1₂ on the high potential side of the output unit 2-2, and connects the electrode 1-14 to the output end P2₂ on the low potential side of the output unit 2-2.

Thereby, the one electrode 1-9 creates a positive electrode, and the one electrode 1-14 creates a negative electrode. That is, an electrode pair including the electrode 1-9 as a positive electrode and the electrode 1-14 as a negative electrode is created between the output end P1₁ on the high potential side and the output end P2₁ on the low potential side of the output unit 2-1, and a current flows to the electrode pair through the user body. This changes a feeling given to the user.

As apparent from the specific examples 1 and 2, the electrical stimulation apparatus 100 according to the present embodiment dynamically switches the combination of the electrodes 1 constituting the electrode pair connected between the output end P1 (P1₁, P1₂) on the high potential side and the output end P2 (P2₁, P2₂) on the low potential side of the output unit 2 (2-1, 2-2). Thus, the electrical stimulation apparatus 100 according to the present embodiment is capable of freely changing the number of electrodes connected to the output unit 2 (2-1, 2-2) (effective electrode area) and the positions of the electrodes connected to the output unit 2 (2-1, 2-2) (effective electrode arrangement). For example, the penetration depth and the range of an applied electrical signal into the body can be changed by changing the arrangement of the electrodes, specifically, the distance between the electrodes to which a current flows. Further, the shape of the electrodes connected to the output unit 2 can also be changed by changing the number and the positions of the electrodes connected to the output unit 2. Thus, according to the present embodiment, tactile presentation can be diversified in the field of tactile presentation via electrical stimulation.

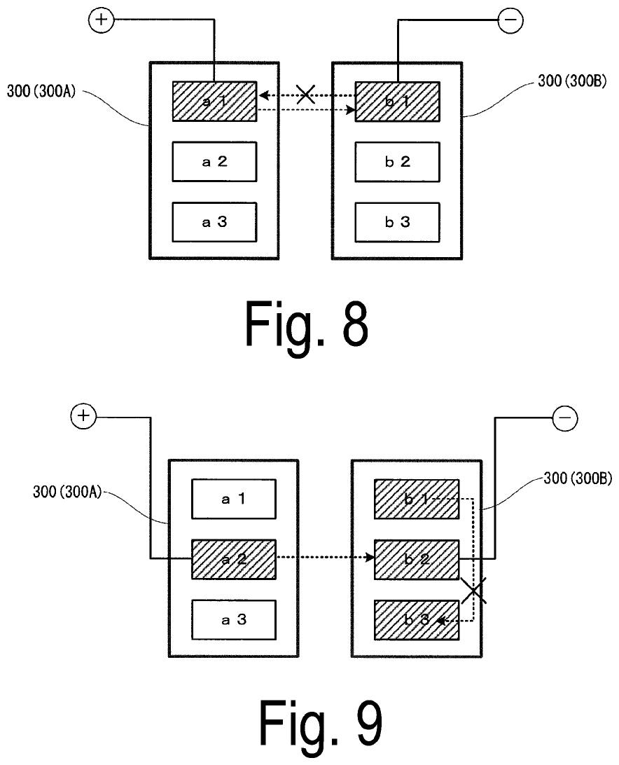

In the embodiment described above, the number of the electrodes 1 is 16 and the number of the output units 2 is two. However, embodiments of the present invention are not limited thereto, and one output unit 2 may be provided. By increasing the number of the electrodes 1 and the number of the output units 2, various types of control are enabled. However, a closed loop cannot be formed unless the electrodes 1 are used in pairs, and thus no current flows to the user. Therefore, given that the number of electrodes 1 is e and the number of output units 2 is d, it is preferable to satisfy e 2d. In the present embodiment, according to an instruction from the control unit 4 to the output selection unit 3, the current flow from the electrode b1 to the electrode a1 in FIG. 8 can be produced, or the current flow from the electrode b1 to the electrode b3 in FIG. 9 can be produced. In addition, by providing an instruction from the control unit 4 to the output selection unit 3, the electrode pair connected to the output unit 2 can be switched at high speed with the electrodes 1-1 to 1-16 attached.

Further, in the embodiment described above, the output end P1 of the output unit 2 is the output end on the high potential side, and the output end P2 of the output unit 2 is the output end on the low potential side. However, the polarity of the output end P1 and P2 of the output unit 2 may be switched. For example, at a certain timing, the output end P1 of the output unit 2 may be set at a high potential and the output end P2 of the output unit 2 may be set at a low potential, and at another timing, the output end P1 of the output unit 2 may be set at a low potential and the output end P2 of the output unit 2 may be set at a high potential. In this manner, the output ends P1 and P2 can output a bipolar potential (AC signal).

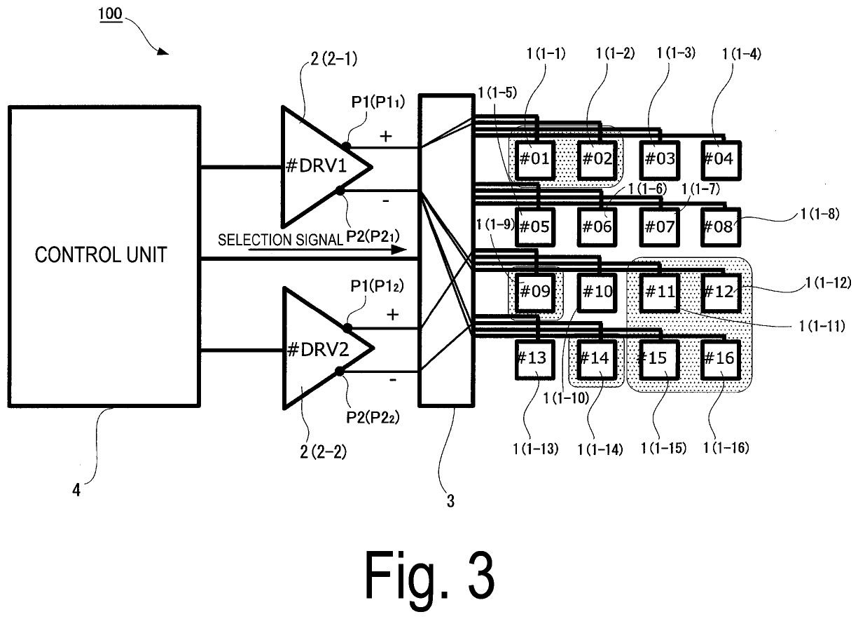

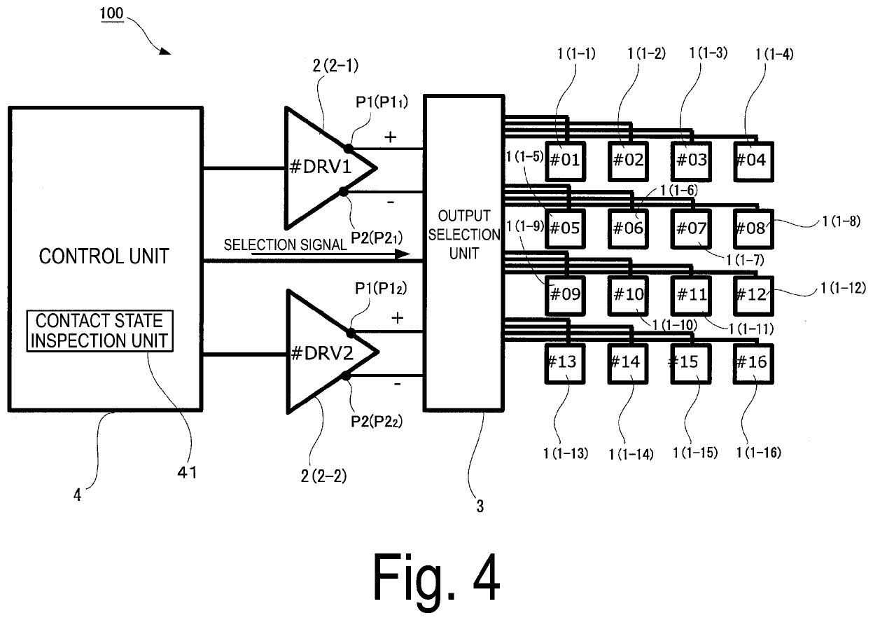

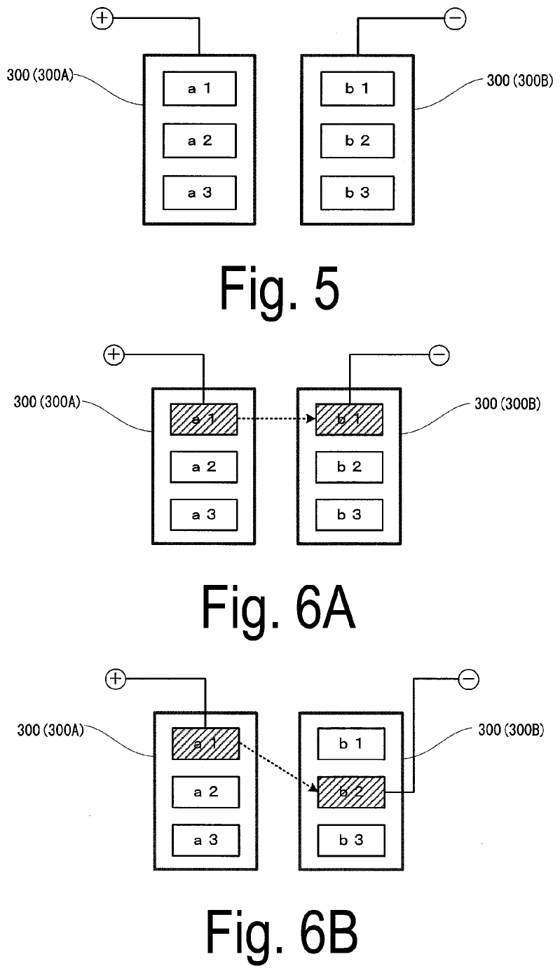

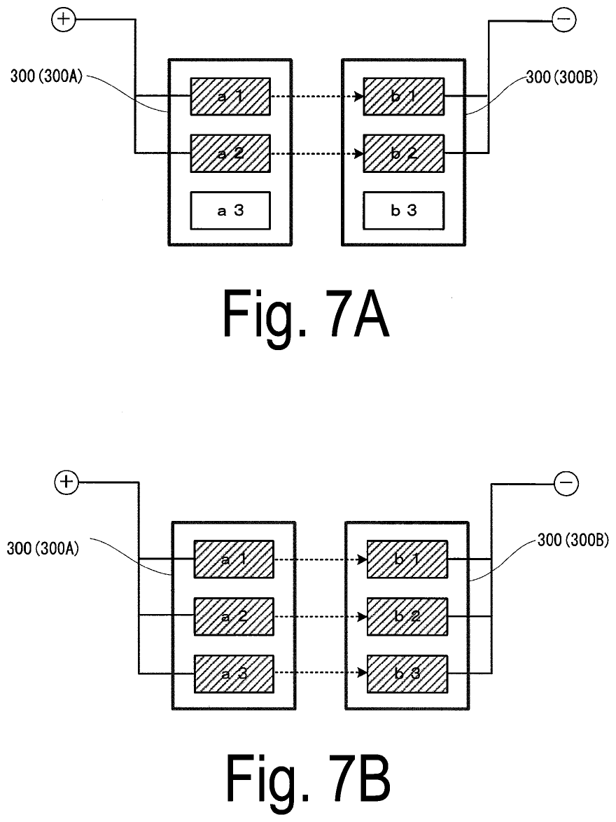

Further, in the embodiment described above, the control unit 4 may be provided with a function unit for inspecting the contact state between each electrode 1 and the place (skin) where the electrode 1 is disposed. FIG. 4 illustrates an example in which the control unit 4 is provided with a contact state inspection unit 41 as the function unit for inspecting the contact state between each electrode 1 and the skin.

This contact state inspection unit 41 is provided as one of the functions implemented by cooperation of the hardware and the program in the control unit 4, and executes processing of inspecting the contact state between each of the electrodes 1-1 to 1-16 and the skin, and excluding the electrode determined to be in a poor contact state from the combination of the electrodes indicated to the output selection unit 3.

The contact state between each electrode 1 and the skin can be recognized by passing a weaker current than usual, which cannot be felt by the user. For example, the contact state inspection unit 41 sends a command to pass a weak current to the output unit 2-1. In addition, the contact state inspection unit 41 sends, to the output selection unit 3, an instruction to sequentially switch the combination of the electrode pair formed between the output end P1₁ on the high potential side and the output end P2₁ on the low potential side of the output unit 2-1, for example, in the order of “#01-#02”, “#02-#03”, . . . “#15-#16,” “#16-#01”.

The contact state inspection unit 41 acquires, from the output unit 2-1, the amount of current at each time of switching of the combination of the electrode pair in the order of “#oi-#02”, “#02-#03”, . . . “#15-#16,” “#16-#01”, and determines the contact state between each of the electrodes 1-1 to 1-16 and the skin based on each of the acquired amounts of current.

In this case, when determining that the contact state is poor, the contact state inspection unit 41 minutely inspects the vicinity thereof. The pair of electrodes 1 forms one closed circuit, and thus, when the electrode 1-2 has a problem, problems should occur between the electrodes 1-1 and 1-2, and between the electrodes 1-2 and 1-3. Thus, it is found out that at a place having the problem the contact state between the electrode and the skin is poor and unsuitable for use.

When there is an electrode determined to be in the poor contact state with the skin, the contact state inspection unit 41 excludes the electrode from the combination of the electrodes indicated to the output selection unit 3. This can prevent selection of the electrode that is not in contact with the skin.

### Expansion of Embodiment

The present invention has been described above with reference to the embodiment, but the present invention is not limited to the above-described embodiment. Various changes that can be understood by a person skilled in the art within the scope of the present invention can be made to the configuration and details of the present invention.

### REFERENCE SIGNS LIST

1 (1-1 to 1-16) Electrode
2 (2-1, 2-2) Output unit
3 Output selection unit
4 Control unit
41 Contact state inspection unit
P1 (P1₁, P1₂) Output end on a high potential side
P2 (P2₁, P2₂) Output end on a low potential side
100 Electrical stimulation apparatus.

1.-5. (canceled)

6. An electrical stimulation apparatus comprising:
a plurality of electrodes;
one or more outputs, each of the one or more outputs including a first output end and a second output end, the first output end and the second output end being different in potential; and
an output selector configured to freely switch a combination of electrodes constituting an electrode pair connected between a respective first output end and a respective second output end of a first output of the one or more outputs.

7. The electrical stimulation apparatus according to claim 6, further comprising:
a controller configured to indicate, to the output selector, the combination of the electrodes constituting the electrode pair connected between the respective first output end and the respective second output end of the first output.

8. The electrical stimulation apparatus according to claim 7, wherein the controller is configured to change a number of the electrodes connected to the respective first output end of the first output and a number of the electrodes connected to the respective second output end of the first output.

9. The electrical stimulation apparatus according to claim 7, wherein the controller is configured to change positions of the electrodes connected to the respective first output end of the first output and positions of the electrodes connected to the respective second output end of the first output.

10. The electrical stimulation apparatus according to claim 7, wherein the controller includes a contact state inspector configured to inspect a contact state between each of the plurality of electrodes and a place where a respective one of the plurality of electrodes is disposed, and exclude an electrode of the plurality of electrodes that is determined to be in a poor contact state from the combination of the electrodes indicated to the output selector.

11. An electrical stimulation apparatus comprising:
a plurality of electrodes;
an output comprising a first output end and a second output end, the first output end and the second output end being different in potential;
an output selector configured to switch a combination of the plurality of electrodes constituting an electrode pair connected between the first output end and the second output end; and
a controller configured to indicate, to the output selector, the combination of the plurality of electrodes constituting the electrode pair.

12. The electrical stimulation apparatus according to claim 11, wherein the controller is configured to change a number of the plurality of electrodes connected to the first output end and a number of the plurality of electrodes connected to the second output end.

13. The electrical stimulation apparatus according to claim 11, wherein the controller is configured to change positions of the plurality of electrodes connected to the first output end and positions of the plurality of electrodes connected to the second output end.

14. The electrical stimulation apparatus according to claim 11, wherein the controller includes a contact state inspector configured to:
inspect a contact state between each of the plurality of electrodes and a place where a respective one of the plurality of electrodes is disposed; and
exclude an electrode of the plurality of electrodes that is determined to be in a poor contact state from the combination of the plurality of electrodes indicated to the output selector.

15. A method comprising:
indicating, by a controller of an electrical stimulation apparatus, a combination of a plurality of electrodes constituting an electrode pair to be connected between a first output end and a second output end of an output, wherein the first output end and the second output end are different in potential, and wherein the electrical stimulation apparatus comprises the output and the plurality of electrodes; and
switching, by an output selector of the electrical stimulation apparatus, the combination of the plurality of electrodes constituting the electrode pair as indicted by the controller.

16. The method according to claim 15, further comprising changing a number of the plurality of electrodes connected to the first output end and a number of the plurality of electrodes connected to the second output end.

17. The method according to claim 15, further comprising changing positions of the plurality of electrodes connected to the first output end and positions of the plurality of electrodes connected to the second output end.

18. The method according to claim 15, further comprising:
inspecting a contact state between each of the plurality of electrodes and a place where a respective one of the plurality of electrodes is disposed; and
excluding an electrode of the plurality of electrodes that is determined to be in a poor contact state from the combination of the plurality of electrodes.
